(19) United States
(12) Patent Application Publication
(10) Pub. No.: US 2025/0109276 A1
(43) Pub. Date: Apr. 3, 2025

(54) STORAGE AND/OR TRANSPORT OF ETHYLENICALLY UNSATURATED COMPOUNDS

(71) Applicant: BASF SE, Ludwigshafen am Rhein (DE)

(72) Inventors: Jan Niclas GORGES, Ludwigshafen am Rhein (DE); Tile GIESHOFF, Ludwigshafen am Rhein (DE); Peter ZUROWSKI, Ludwigshafen am Rhein (DE); Friederike FLEISCHHAKER, Ludwigshafen am Rhein (DE); Rebecca SURE, Ludwigshafen am Rhein (DE); Christian REIN, Ludwigshafen am Rhein (DE); Friedrich-Georg MARTIN, Ludwigshafen am Rhein (DE)

(21) Appl. No.: 18/832,953

(22) PCT Filed: Jan. 16, 2023
(86) PCT No.: PCT/EP2023/050806

§ 371 Date: Jul. 25, 2024

(30) Foreign Application Priority Data
Jan. 26, 2022 (EP) 22153375.5

(51) Int. Cl. C08K 5/3435; C08F 20/06; C08F 20/14; C08F 20/18

(52) U.S. Cl. CPC C08K 5/3435; C08F 20/06; C08F 20/14; C08F 20/18

(57) ABSTRACT

The present invention relates to a method of storage and/or transport of an ethylenically unsaturated compound, wherein the ethylenically unsaturated compound is protected against undesired free-radical polymerization with a sterically hindered N-oxyl.

The present invention relates to a method of storage and/or transport of an ethylenically unsaturated compound, wherein the ethylenically unsaturated compound is protected against undesired free-radical polymerization with a sterically hindered N-oxyl.

Chemical compounds having one or more ethylenically unsaturated groups have a pronounced tendency to free-radical polymerization. Such compounds are therefore subsequently also referred to as polymerizable compounds. The tendency of these compounds to free-radical polymerization means that they are used as monomers for the production of polymers. However, the pronounced tendency to free-radical polymerization of these compounds is also a dis-advantage in that undesired spontaneous free-radical polymerization can occur during storage and transport and also during chemical and/or physical processing such as distillation or rectifi-cation, particularly under the action of energy such as heat and/or light. Such uncontrolled polymerizations can result in the gradual formation of polymer deposits, for example on heated surfaces, that necessitates removal of the polymer deposits and thus often results in shortened operating times. The uncontrolled polymerizations may even proceed explosively.

During storage and transport and also during chemical and/or physical processing of ethylenically unsaturated compounds that have a tendency to free-radical polymerization or mixtures that comprise such compounds, it is therefore customary to add compounds that prevent or at least retard undesired spontaneous free-radical polymerization. Such substances are referred to as polymerization inhibitors.

Polymerization inhibitors may be employed as individual chemical compounds or as mixtures of compounds. Particular requirements are placed on the polymerization inhibitor, depending on its field of use. For a polymerization inhibitor to be suitable as a transport and/or storage stabilizer of ethylenically unsaturated compounds it is important that the efficiency of the polymerization inhibitor, i.e. the extent of inhibition of polymerization, is controllable. Under the conditions of storage and/or transport of the ethylenically unsaturated compounds, the polymerization inhibitor should adequately prevent or retard the undesired spontaneous free-radical polymerization, whereas the desired free-radical polymerization of the ethylenically unsaturated compounds should be possible under appropriate polymerization conditions without the need to first have to separate the polymerization inhibitor used during storage and/or transport. If the stabilizer used during storage and/or transport is not separated for the desired polymerization, it is important that it does not adversely affect the desired polymerization, for example does not unintentionally act as a chain-transfer agent.

In terms of global production volume, acrylic acid is without doubt one of the most important ethylenically unsaturated compounds. During storage and/or transport, acrylic acid is normally stabilized against undesired spontaneous free-radical polymerization with 0.018% to 0.022% by weight of hydroquinone monomethyl ether (MEHQ), based on the amount of acrylic acid. For adequate stabilization of acrylic acid against undesired spontaneous free-radical polymerization using MEHQ, it is necessary that oxygen is dissolved in the acrylic acid in sufficient amounts. Sufficient amounts of oxygen are generally dissolved in the acrylic acid when acrylic acid is stored and/or transported under an atmosphere having 5% to 21% by volume of oxygen. In the desired polymerization of acrylic acid the content of dissolved oxygen in the acrylic acid is de-creased, thus reducing the efficiency of MEHQ as a polymerization inhibitor enough for acrylic acid to undergo polymerization in the presence of MEHQ.

In addition to use as a polymerization inhibitor for the storage and/or transport of acrylic acid, MEHQ is also used as a polymerization inhibitor for the storage and/or transport of methacrylic acid, acrylic esters and/or methacrylic esters or of mixtures comprising one or more of said compounds.

Its widespread use makes MEHQ one of the most important storage and/or transport stabilizers for ethylenically unsaturated compounds, especially for acrylic acid, methacrylic acid, acrylic esters and methacrylic esters.

Ethylenically unsaturated compounds are stored in permanently installed vessels that are suitable for the purpose, such as storage tanks (see for example Acrylic Acid, A Summary of Safety and Handling, 4th edition 2013, 7 Bulk Storage Facilities and Accessories). Preferably, the storage of the ethylenically unsaturated compounds in the respective vessels is preferably for at least one hour, more preferably at least 10 hours, most preferably at least 100 hours. Although the duration of storage under appropriate conditions is theoretically unlimited, the duration of storage is generally reduced to a minimum for economic reasons. Preferably, the storage of the ethylenically unsaturated compounds in the respective vessels is for not more than 180 days, more preferably for not more than 90 days, most preferably for not more than 30 days. Particularly preferred ranges result from the free combination of the aforementioned lower and upper ranges.

Ethylenically unsaturated compounds are typically transported in suitable transportable vessels, such as tanks or drums, by ship, rail and/or truck (see for example Acrylic Acid, A Summary of Safety and Handling, 4th edition 2013, 9 Safe Transport of Acrylic Acid). The transportable vessels may of course also be permanently installed on the corresponding means of transportation, for example tanker ships or rail tank cars. It is of course also possible for vessels to be stored for a certain time in a particular place before they are transported to another site. Ethylenically unsaturated compounds may also be transported in pipelines or hoses, for example to a storage tank after purification of the ethylenically unsaturated compound, in the case of transfer from the storage tank into a transportable vessel, and/or in the case of transfer from one transportable vessel into another. Preferably, the transportation of the ethylenically unsaturated compounds in the respective vessels is preferably for at least one hour, more preferably at least 10 hours, most preferably at least 100 hours. Although the duration under appropriate conditions is theoretically unlimited, the duration is generally reduced to a minimum for economic and safety reasons. Preferably, the transport of the ethylenically unsaturated compounds in the respective vessels is for not more than 180 days, more preferably for not more than 90 days, most preferably for not more than 30 days. Particularly preferred ranges result from the free combination of the aforementioned lower and upper ranges.

EP 0 685 447 A2 and JP 2002-234858 A disclose mixtures of polymerization inhibitors for (meth)acrylic acid and esters thereof. The mixtures comprise N-oxyls.

WO 2001/040149 A2 and WO 2001/040319 A2 disclose mixtures of polymerization inhibitors for ethylenically unsaturated monomers. The mixtures likewise comprise N-oxyls.

It was an object of the present invention to provide a method of storage and/or transport of ethylenically unsaturated compounds. The polymerization inhibitors are to ensure sufficient stabilization of the ethylenically unsaturated compounds against undesired free-radical polymerization. In the desired free-radical polymerization, however, there should be no need to separate the polymerization inhibitors from the mixture before the polymerization. The polymerization inhibitors are therefore not to act as polymerization chain-transfer agents and/or polymerization inhibitors in the desired free-radical polymerization.

The object is achieved by a method of storage and/or transport of an ethylenically unsaturated compound, where the ethylenically unsaturated compound has a purity of at least 90% by weight, the mass of stored and/or transported ethylenically unsaturated compound is at least 100 kg, the ethylenically unsaturated compound has a temperature during storage and/or transport of less than 50° C. and the ethylenically unsaturated compound is a carboxylic acid or a carboxylic ester, wherein the ethylenically unsaturated compound comprises from 0.00001% to 0.00050% by weight (0.1 to 5 ppm), based on the total amount of ethylenically unsaturated compound, of a sterically hindered N-oxyl.

Sterically hindered N-oxyls have an NO group with a free electron on the oxygen atom and fully substituted carbon atoms aside from the NO group.

Suitable sterically hindered N-oxyls are 1-oxyl-2,2,6,6-tetramethylpiperidine, 1-oxyl-2,2,6,6-tet-ramethylpiperidin-4-ol (HTEMPO), 1-oxyl-2,2,6,6-tetramethylpiperidin-4-one, 1-oxyl-2,2,6,6-tet-ramethylpiperidin-4-yl acetate, 1-oxyl-2,2,6,6-tetramethylpiperidin-4-yl 2-ethylhexanoate, 1-oxyl-2,2,6,6-tetramethylpiperidin-4-yl stearate, 1-oxyl-2,2,6,6-tetramethylpiperidin-4-yl benzoate, 1-oxyl-2,2,6,6-tetramethylpiperidin-4-yl (4-tert-butyl)benzoate, bis(1-oxyl-2,2,6,6-tetramethylpiperi-din-4-yl) succinate, bis(1-oxyl-2,2,6,6-tetramethylpiperidin-4-yl) adipate, bis(1-oxyl-2,2,6,6-tetra-methylpiperidin-4-yl) sebacate, bis(1-oxyl-2,2,6,6-tetramethylpiperidin-4-yl) n-butylmalonate, bis(1-oxyl-2,2,6,6-tetramethylpiperidin-4-yl) phthalate, bis(1-oxyl-2,2,6,6-tetramethylpiperidin-4-yl) isophthalate, bis(1-oxyl-2,2,6,6-tetramethylpiperidin-4-yl) terephthalate, bis(1-oxyl-2,2,6,6-tetramethylpiperidin-4-yl) hexahydroterephthalate, N,N′-bis(1-oxyl-2,2,6,6-tetramethylpiperidin-4-yl)adipamide, N-(1-oxyl-2,2,6,6-tetramethylpiperidin-4-yl)caprolactam, N-(1-oxyl-2,2,6,6-tetra-methylpiperidin-4-yl)dodecylsuccinimide, 2,4,6-tris[N-butyl-N-(1-oxyl-2,2,6,6-tetramethylpiperi-din-4-yl]-s-triazine and tris(1-oxyl-2,2,6,6-tetramethylpiperidin-4-yl) phosphite.

The purity of the ethylenically unsaturated compound is preferably at least 95% by weight, more preferably at least 98% by weight, most preferably at least 99% by weight.

The mass of stored and/or transported ethylenically unsaturated compound is preferably at least 200 kg, more preferably at least 500 kg, most preferably at least 1000 kg. The mass of stored and/or transported ethylenically unsaturated compound is typically less than 10 000 000 kg.

The ethylenically unsaturated compound has a temperature during storage and/or transport of preferably less than 45° C., more preferably less than 40° C., most preferably less than 35° C. In the case of acrylic acid, the temperature during storage and/or transport should be at least 15° C.

The ethylenically unsaturated compound preferably comprises less than 0.0001% by weight of each of a manganese salt, a copper salt, a 2,2,6,6-tetramethylpiperidine compound that is not an N-oxyl, and a nitroso compound, based in each case on the total amount of ethylenically unsaturated compound.

In a preferred embodiment of the present invention, the sterically hindered N-oxyl is used essentially as the sole polymerization inhibitor.

The ethylenically unsaturated compound preferably comprises from 0.00005% to 0.00045% by weight (0.5 to 4.5 ppm), more preferably from 0.00010% to 0.00040% by weight (1 to 4 ppm), most preferably from 0.00015% to 0.00035% by weight (1.5 to 3.5 ppm), based in each case on the total amount of ethylenically unsaturated compound.

The ethylenically unsaturated compounds are preferably mono-, di- or triethylenically unsaturated C₃- to C₈-carboxylic acids, mono-, di- or triethylenically unsaturated C₃- to C₈-carboxylic esters having 1 to 20 carbon atoms in the ester groups.

Particular preference is given to mono-, di- or triethylenically unsaturated C₃- to C₆-carboxylic acids, for example acrylic acid, methacrylic acid, dimethacrylic acid, ethacrylic acid, citraconic acid, methylenemalonic acid, crotonic acid, fumaric acid, mesaconic acid, itaconic acid and maleic acid, mono-, di- or triethylenically unsaturated C₃- to C₆-carboxylic esters having 1 to 12 carbon atoms in the ester groups, for example acrylic esters with C₁- to C₁₂-alkyl, methacrylic esters with C₁- to C₁₂-alkyl, dimethacrylic esters with C₁- to C₁₂-alkyl, ethacrylic esters with C₁- to C₁₂-alkyl, citraconic esters with C₁- to C₁₂-alkyl, methylenemalonic esters with C₁- to C₁₂-alkyl, crotonic esters with C₁- to C₁₂-alkyl, fumaric esters with C₁- to C₁₂-alkyl, mesaconic esters with C₁- to C₁₂-alkyl, itaconic esters with C₁- to C₁₂-alkyl and maleic esters with C₁- to C₁₂-alkyl.

Most particular preference is given to acrylic acid, methacrylic acid, acrylic esters with C₁- to C₈-alkyl, such as methyl acrylate, ethyl acrylate, n-butyl acrylate, and 2-ethylhexyl acrylate, and methacrylic esters with C₁- to C₈-alkyl, such as methyl methacrylate.

Further suitable ethylenically unsaturated compounds are dipropylene glycol diacrylate, tripro-pylene glycol diacrylate, polyethylene glycol diacrylate, glycerol triacrylate, ethoxylated glycerol triacrylate, trimethylolpropane triacrylate, ethoxylated trimethylolpropane triacrylate, butanediol monoacrylate, dicyclopentadienyl acrylate, 2-dimethylaminoethyl acrylate, 2-hydroxyethyl acrylate, and 2-hydroxypropyl acrylate.

Acrylic acid and/or methacrylic acid are typically stored and/or transported in stainless steel vessels. Suitable stainless steels comprise 16.5% to 19.5% by weight of chromium and from 8.0% to 13.5% by weight of nickel.

The ethylenically unsaturated compound is typically stored and/or transported under an oxygen-containing atmosphere.

The ethylenically unsaturated compound is preferably stored in a vessel under an oxygen-containing atmosphere having an oxygen content of 5% to 10% by volume and the mixture in the vessel is regularly recirculated, for example by pumped recirculation of the entirety of the tank contents at least once a week. The relatively low oxygen content prevents the formation of ignit-able gas mixtures in the vessel. The recirculation replaces consumed dissolved oxygen in the liquid ethylenically unsaturated compound.

The present invention further provides a method of free-radical polymerization, wherein at least one of the above-described ethylenically unsaturated compounds is polymerized by means of a polymerization initiator, and for the use of one of the above-described ethylenically unsaturated compounds for free-radical polymerization by means of a polymerization initiator.

By the free-radical polymerization of the invention, for example, water-soluble and water-swella-ble polyacrylic acids and sodium salts thereof are obtainable.

Free-radical polymerization itself is common knowledge and is typically conducted in solution. Suitable polymerization initiators are all compounds that can break down to free radicals under the chosen reaction conditions, for example thermal initiators, redox initiators, photoinitiators. Suitable thermal initiators are peroxomono- and -disulfates, and peroxomono- and -diphos-phates. Suitable redox initiators are sodium peroxodisulfate/ascorbic acid, hydrogen perox-ide/ascorbic acid, sodium peroxodisulfate/sodium hypophosphite, sodium peroxodisulfate/sodium bisulfite and hydrogen peroxide/sodium bisulfite.

### EXAMPLES

### Inhibition of Polymerization Experiments

The ethylenically unsaturated compound used was distilled twice to remove the hydroquinone monomethyl ether (MEHQ) polymerization inhibitor. The stated amount of the specified polymerization inhibitor was added in each case to the ethylenically unsaturated compound obtained. MEHQ and 4-hydroxy-2,2,6,6-tetramethylpiperidine N-oxyl (HTEMPO) were used.

0.5 ml of each mixture was transferred to a 1.8 ml ampoule and stored at the specified temperature in an air circulation oven.

In each test series, in each case three ampoules were filled with each mixture and tested, with the average time for complete polymerization determined by visual examination.

TABLE 1
Mixtures comprising acrylic acid

| Polymerization | | | Time before | |
|---|---|---|---|---|
| inhibitor | Amount | Temperature | polymerization | |
| none*) | | 100° C. | 0.95 | h |
| MEHQ*) | 0.0005% by wt. | 100° C. | 1.31 | h |
| MEHQ*) | 0.0010% by wt. | 100° C. | 1.87 | h |
| MEHQ*) | 0.0020% by wt. | 100° C. | 2.43 | h |
| MEHQ*) | 0.0025% by wt. | 100° C. | 2.85 | h |
| MEHQ*) | 0.0040% by wt. | 100° C. | 3.92 | h |
| MEHQ*) | 0.0050% by wt. | 100° C. | 4.25 | h |
| MEHQ*) | 0.0060% by wt. | 100° C. | 4.99 | h |
| MEHQ*) | 0.0100% by wt. | 100° C. | 6.75 | h |
| HTEMPO | 0.00005% by wt. | 100° C. | 1.48 | h |
| HTEMPO | 0.0001% by wt. | 100° C. | 2.95 | h |
| HTEMPO | 0.00015% by wt. | 100° C. | 3.93 | h |
| HTEMPO | 0.0002% by wt. | 100° C. | 7.17 | h |
| HTEMPO | 0.00025% by wt. | 100° C. | 10.18 | h |
| HTEMPO | 0.00035% by wt. | 100° C. | >14 | h |
| HTEMPO | 0.00045% by wt. | 100° C. | >14 | h |

*)comparative

TABLE 2
Mixtures comprising methacrylic acid

| Polymerization | | | Time before |
|---|---|---|---|
| inhibitor | Amount | Temperature | polymerization |
| none*) | | 100° C. | 0.83 h |
| MEHQ*) | 0.0005% by wt. | 100° C. | 2.07 h |
| MEHQ*) | 0.0010% by wt. | 100° C. | 3.07 h |
| MEHQ*) | 0.0020% by wt. | 100° C. | 4.54 h |
| MEHQ*) | 0.0040% by wt. | 100° C. | 8.80 h |
| MEHQ*) | 0.0060% by wt. | 100° C. | 11.24 h |
| HTEMPO | 0.00015% by wt. | 100° C. | 2.38 h |
| HTEMPO | 0.00025% by wt. | 100° C. | 4.05 h |
| HTEMPO | 0.00035% by wt. | 100° C. | 4.90 h |
| HTEMPO | 0.00045% by wt. | 100° C. | 5.93 h |

*)comparative

TABLE 3
Mixtures with methyl acrylate

| Polymerization | | | Time before |
|---|---|---|---|
| inhibitor | Amount | Temperature | polymerization |
| none*) | | 120° C. | 0.44 h |
| MEHQ*) | 0.0005% by wt. | 120° C. | 0.59 h |
| MEHQ*) | 0.0010% by wt. | 120° C. | 0.66 h |
| MEHQ*) | 0.0020% by wt. | 120° C. | 0.79 h |
| HTEMPO | 0.00015% by wt. | 120° C. | 0.69 h |
| HTEMPO | 0.00025% by wt. | 120° C. | 0.87 h |
| HTEMPO | 0.00035% by wt. | 120° C. | 0.97 h |
| HTEMPO | 0.00045% by wt. | 120° C. | 1.09 h |

*)comparative

TABLE 4
Mixtures with ethyl acrylate

| Polymerization | | | Time before |
|---|---|---|---|
| inhibitor | Amount | Temperature | polymerization |
| none*) | | 120° C. | 0.51 h |
| MEHQ*) | 0.0005% by wt. | 120° C. | 0.90 h |
| MEHQ*) | 0.0010% by wt. | 120° C. | 1.53 h |
| MEHQ*) | 0.0020% by wt. | 120° C. | 2.62 h |
| HTEMPO | 0.00015% by wt. | 120° C. | 1.08 h |
| HTEMPO | 0.00025% by wt. | 120° C. | 1.48 h |
| HTEMPO | 0.00035% by wt. | 120° C. | 2.23 h |
| HTEMPO | 0.00045% by wt. | 120° C. | 2.59 h |

*)comparative

TABLE 5
Mixtures with n-butyl acrylate

| Polymerization | | | Time before |
|---|---|---|---|
| inhibitor | Amount | Temperature | polymerization |
| none*) | | 120° C. | 0.42 h |
| MEHQ*) | 0.0005% by wt. | 120° C. | 0.58 h |
| MEHQ*) | 0.0010% by wt. | 120° C. | 0.72 h |
| MEHQ*) | 0.0020% by wt. | 120° C. | 1.07 h |
| HTEMPO | 0.00015% by wt. | 120° C. | 0.88 h |
| HTEMPO | 0.00025% by wt. | 120° C. | 1.23 h |
| HTEMPO | 0.00035% by wt. | 120° C. | 1.66 h |
| HTEMPO | 0.00045% by wt. | 120° C. | 2.09 h |

*)comparative

TABLE 6
Mixtures with 2-ethylhexyl acrylate

| Polymerization | | | Time before |
|---|---|---|---|
| inhibitor | Amount | Temperature | polymerization |
| none*) | | 120° C. | 0.95 h |
| MEHQ*) | 0.0005% by wt. | 120° C. | 1.01 h |
| MEHQ*) | 0.0010% by wt. | 120° C. | 1.46 h |
| MEHQ*) | 0.0020% by wt. | 120° C. | 3.44 h |
| HTEMPO | 0.00015% by wt. | 120° C. | 2.13 h |
| HTEMPO | 0.00025% by wt. | 120° C. | 3.06 h |
| HTEMPO | 0.00035% by wt. | 120° C. | 4.58 h |
| HTEMPO | 0.00045% by wt. | 120° C. | 6.09 h |

*)comparative

4-Hydroxy-2,2,6,6-tetramethylpiperidine N-oxyl (HTEMPO), even in a distinctively smaller con-centration, has a comparable inhibiting effect to MEHQ.

### Polymerization Experiments

A reaction vessel was initially charged under a nitrogen atmosphere with 450 g of water. The initially charged reaction mixture was heated to 95° C. with stirring. On attainment of the temperature of 95° C., three streams were metered in with stirring and while maintaining the temperature.
Stream 1: 500 g of acrylic acid, metered in over 5 h
Stream 2: 15 g of sodium hypophosphite in 35 g of deionized water, metered in over 4.75 h
Stream 3: 5 g of sodium peroxodisulfate in 66.4 g of deionized water, metered in over 5.25 h

After addition of the three streams, the reaction mixture was stirred at 95° C. for a further hour. The reaction mixture was then cooled to room temperature and 80 g of water was added.

The acrylic acid used in stream 1 was stabilized with MEHQ or 4-hydroxy-2,2,6,6-tetramethylpiperidine N-oxyl (HTEMPO).

The polymers obtained were analyzed by GPC (calibration with Na-PAA standard, eluent: 0.01 mol/l phosphate buffer pH=7.4 in distilled water containing 0.01 M NaN₃).

TABLE 7
Polymerization of acrylic acid

| Polymer- | | Number | Weight | Poly- |
|---|---|---|---|---|
| ization | | average | average | dispersity |
| inhibitor | Amount | Mn | Mw | Mw/Mn |
| MEHQ*) | 0.020% by wt. | 3240 g/mol | 11 100 g/mol | 3.4 |
| HTEMPO | 0.0002% by wt. | 3190 g/mol | 12 100 g/mol | 3.3 |
| HTEMPO | 0.0005% by wt. | 3320 g/mol | 11 600 g/mol | 3.5 |

*)comparative

4-Hydroxy-2,2,6,6-tetramethylpiperidine N-oxyl (HTEMPO) with the concentrations examined leads to polyacrylic acids having a comparable molar mass to that when the standard concen-tration of MEHQ is used.

1.-15. (canceled)

16. A method of storage and/or transport of an ethylenically unsaturated compound, where the ethylenically unsaturated compound has a purity of at least 90% by weight, the mass of stored and/or transported ethylenically unsaturated compound is at least 100 kg, the ethylenically unsaturated compound has a temperature during storage and/or transport of less than 50° C. and the ethylenically unsaturated compound is a carboxylic acid or a carboxylic ester, wherein the ethylenically unsaturated compound comprises from 0.00001% to 0.00050% by weight, based on the total amount of ethylenically unsaturated compound, of a sterically hindered N-oxyl.

17. The method according to claim 16, wherein the ethylenically unsaturated compound comprises less than 0.0001% by weight of each of a manganese salt, a copper salt, a 2,2,6,6-tetramethylpiperidine compound that is not an N-oxyl, and a nitroso compound, based in each case on the total amount of ethylenically unsaturated compound.

18. The method according to claim 16, wherein the sterically hindered N-oxyl is used essentially as the sole polymerization inhibitor.

19. The method according to claim 16, wherein 1-oxyl-2,2,6,6-tetramethylpiperidin-4-ol is the sterically hindered N-oxyl used.

20. The method according to claim 16, wherein storage and/or transport of the ethylenically unsaturated compound extends over a period of at least 10 hours.

21. The method according to claim 16, wherein the ethylenically unsaturated compound comprises from 0.00005% to 0.00045% by weight, based on the total amount of ethylenically unsaturated compound, of a sterically hindered N-oxyl.

22. The method according to claim 16, wherein the ethylenically unsaturated compound comprises from 0.00010% to 0.00040% by weight, based on the total amount of ethylenically unsaturated compound, of a sterically hindered N-oxyl.

23. The method according to claim 16, wherein the ethylenically unsaturated compound comprises from 0.00015% to 0.00035% by weight, based on the total amount of ethylenically unsaturated compound, of a sterically hindered N-oxyl.

24. The method according to claim 16, wherein the ethylenically unsaturated compound is acrylic acid, methacrylic acid, methyl acrylate, methyl methacrylate, ethyl acrylate, n-butyl acrylate and/or 2-ethylhexyl acrylate.

25. The method according to claim 16, wherein the ethylenically unsaturated compound is stored and/or transported under an oxygen-containing atmosphere.

26. The method according to claim 16, wherein the ethylenically unsaturated compound is stored in a vessel under an oxygen-containing atmosphere having an oxygen content of 5% to 10% by volume and the ethylenically unsaturated compound in the vessel is regularly recirculated.

27. A method of free-radical polymerization, where at least one ethylenically unsaturated compound is polymerized by means of a polymerization initiator and the ethylenically unsaturated compound is a carboxylic acid or a carboxylic ester, wherein the ethylenically unsaturated compound used has a purity of at least 90% by weight and the ethylenically unsaturated compound comprises from 0.00001% to 0.00050% by weight, based on the total amount of ethylenically unsaturated compound, of a sterically hindered N-oxyl.

28. The method according to claim 27, wherein the sterically hindered N-oxyl is used essentially as the sole polymerization inhibitor.

29. A use of an ethylenically unsaturated carbonyl compound for free-radical polymerization by means of a polymerization initiator, wherein the ethylenically unsaturated compound is a carboxylic acid or a carboxylic ester, the ethylenically unsaturated compound has a purity of at least 90% by weight and the ethylenically unsaturated compound comprises from 0.00001% to 0.00050% by weight, based on the total amount of ethylenically unsaturated compound, of a sterically hindered N-oxyl.

30. The use according to claim 29, wherein the sterically hindered N-oxyl is used essentially as the sole polymerization inhibitor.
